(19) United States
(12) Patent Application Publication
(10) Pub. No.: US 2008/0221693 A1
(43) Pub. Date: Sep. 11, 2008

(54) INTERVERTEBRAL PROSTHESIS

(76) Inventors: Peter Brehm, Weisendorf (DE); Herbert Gluch, Rosenheim (DE)

Correspondence Address: SUMMA, ALLAN & ADDITON, P.A., 11610 NORTH COMMUNITY HOUSE ROAD, SUITE 200, CHARLOTTE, NC, 28277, US

(21) Appl. No.: 12/042,572

(22) Filed: Mar. 5, 2008

(30) Foreign Application Priority Data
Mar. 7, 2007 (DE) 202007003420.2

(51) Int. Cl. A61F 2/44

(52) U.S. Cl. 623/17.16; 623/17.11; 623/17.15

(57) ABSTRACT

An intervertebral prosthesis is disclosed for utilisation as an intervertebral disc replacement in the skeleton of the human body. The intervertebral prosthesis includes a first prosthesis plate, on which is formed a first coupling element, e.g., in the form of a spherical cap, and a second prosthesis plate, in which, for example, a hollow spherical shell is formed, into which, in the coupled state, the spherical cap engages. To create a physiologically favourable intervertebral disc replacement, the rotational movement of both prosthesis plates with respect to each other around an axis perpendicular to the prosthesis plates is significantly limited, for example to 3 degrees. The limitation of the rotational movement is produced by means of a movement restriction means in the form of mechanical blocks on the coupling elements.

### BACKGROUND

The invention relates to an intervertebral prosthesis for utilisation as an intervertebral disc replacement in the skeleton of the human body.

An intervertebral disc prosthesis is known, for example, from EP 1 642 553 A1. The intervertebral disc prosthesis described therein consists of a first prosthesis plate and a second prosthesis plate, as well as a prosthesis core, which is arranged between the first prosthesis plate and the second prosthesis plate and which is formed separately from them. One of the prosthesis plates has a depression in the shape of a spherical shell, whereby a spherical segment of the prosthesis core engages in this depression. The second prosthesis plate has an essentially level depression, into which an essentially flat prosthesis core section, which lies opposite the spherical segment, engages, whereby the level section of the prosthesis core can be slid in a depression in the second prosthesis plate. After the first and second prosthesis plates have been brought into place at the respective vertebra surface adjacent to the intervertebral area, the prosthesis core can be inserted separately. When the prosthesis plates are tilted with respect to each other, a lateral displacement of the prosthesis core in the depression of the second prosthesis plate is possible. Due to the ball-shaped formation of the prosthesis core, unrestricted rotational movement of the first prosthesis plate with respect to the second prosthesis plate around an axis of rotation that is essentially perpendicular to the surfaces of the prosthesis plate is possible.

Body implants, which serve as replacement elements for the body's own defective or pathogenic elements or organs, are intended physiologically to copy, as exactly as possible, the inherent characteristics of the replacement elements or organs. In the case of an intervertebral implant that is intended to serve as an intervertebral disc replacement, the function and formation of the intervertebral disc are therefore to be copied in a manner that is as lifelike as possible, so that functions inherent to the body can be optimally copied by the implant and so that no unphysiological processes are initiated, said unphysiological processes being, as a rule, detrimental to the wellbeing of the patient.

The object of the invention therefore consists in providing an intervertebral prosthesis that comes as close as possible to the physiological function and nature of an intervertebral disk and that does not create any unphysiological processes, in order to avoid new complaints being induced by the implant.

### SUMMARY

This object is solved according to the invention by an intervertebral prosthesis for utilisation as an intervertebral disc replacement in the skeleton of the human body, said intervertebral prosthesis having a first prosthesis plate and a second prosthesis plate, whereby on each of the first and second prosthesis plates, coupling elements with surface elements conjugate to each other are formed, whereby the coupling elements, in the coupled state, allow a rotation as well as a tilting movement in the lateral as well as in the posterior and anterior directions with respect to an axis running essentially perpendicularly to an extension of the first and second plates, whereby the intervertebral prosthesis is characterised in that movement restriction means are present that restrict the rotational movement to a predetermined maximum value.

Due to the movement restriction means for restricting the rotational movement, an intervertebral implant that approximates the human intervertebral disc is created. By restricting the rotational movement in the intervertebral prosthesis according to the invention, excess stresses on the vertebral joints in the human skeleton, which occur with conventional intervertebral disc replacements, are avoided. In this way, a distinct improvement in the stability of the overall motion segment is achieved, and finally the wellbeing of the patient who has been equipped with the intervertebral implant according to the invention is distinctly improved.

In an embodiment, the maximum predetermined value for the rotational movement is an angle of 5 degrees, or of 3 degrees. As a result of this distinct limitation of the movement restriction in the rotation by the intervertebral implant according to the invention, in the case of a torsional movement of the trunk of the patient who has been equipped with the intervertebral implant, the twist that then arises in the spinal column is distributed among a larger number of intervertebral areas and is not only primarily absorbed by one intervertebral area that is unlimited in rotation, so that overstressing of the ligament, muscle and joint complexes in this area can be avoided.

According to a further embodiment, the surface elements of the coupling elements, said surface elements being formed conjugate to each other, are, on the one hand, a spherical cap which is provided on the first prosthesis plate, as well as a matching hollow spherical shell surface, which is placed on the second prosthesis plate. As a result of this formation of the coupling elements, which may be integrally formed on to the respective prosthesis plates, optimal coupling is achieved across the intended maximum movement range, which realises, in the simplest manner, both rotational and tilting movements in all directions by means of a single coupling element.

According to a further embodiment, in the case of this formation of the coupling elements, the movement restriction means is formed as a tab-shaped overlap on the spherical cap, as well as a correspondingly predetermined cut formed in the hollow spherical shell surface, said cut allowing a maximum value for the rotational angle. During the rotational movement of the two prosthesis plates with respect to each other, the tab-shaped overlap formed on the spherical cap restricts the possible rotational movement of the two prosthesis plates in the coupled state due to the fact that the tab-shaped overlap hits against the corresponding matching cut at the end of the allowed predetermined maximum rotation area, said cut, however, being formed to be larger. By means of a suitable selection of the width of the tab-shaped overlap, or suitable selection of the width of the cut, it is possible to restrict the maximum deflection of the rotation to a predetermined value, depending on the size of the spherical cap. By means of the selection of the formation of the height of the tab-shaped overlap, as well as by means of a suitable corresponding selection of the depth of the cut formed in the hollow spherical shell surface, it is likewise additionally possible in the coupled state to restrict to a predetermined value the maximum tilting of the two prosthesis plates with respect to each other in the direction in which the tab-shaped overlap is arranged with respect to the centre point of the spherical cap.

In a further embodiment, two overlaps, which lie opposite each other, are provided, with said overlaps engaging in two corresponding cuts, which lie opposite each other, in the hollow spherical shell surface in the coupled state. In this way, particularly reliable mechanical restriction of the movement is achieved, which then also guarantees with a high degree of certainty the restriction of the rotational movement, should there be, in addition to rotation, a tilting movement of the two prosthesis plates with respect to each other in the form of a bending or stretching of the vertebral joint. In this way, even in the case of the very complicated movements that are possible in the physiological movement sequence and that have to be absorbed by the intervertebral area, reliable restriction of the movement absorption is guaranteed, and, due to this fact, unphysiological movement sequences are reliably avoided.

In a further embodiment, the movement restriction means are formed in such a way that a tilting movement in the coupled state is restricted to maximum values that correspond to a value of 15 degrees in the event of tilting in the posterior or in the anterior direction. The maximum value for the restriction of the lateral tilting movement may be as low as 6 degrees.

In order to allow movements relative to the body axis in both the lateral and in the posterior and anterior directions with the intervertebral prosthesis, with the simple configuration according to the invention, inserted into the human skeleton, the spherical cap is formed so that it is larger than the corresponding hollow spherical shell surface, so that the spherical cap, which is formed on the prosthesis plate, can be tipped across the predetermined angle of tilt against the hollow spherical shell surface formed on the opposing prosthesis plate, before the edge of the hollow spherical shell hits against the opposing prosthesis plate. The front faces of the hollow spherical shell are preferably formed so that they are correspondingly sloped, so that when the front faces of the hollow spherical shell hit, a flat hitting against the opposing prosthesis plate takes place. The front edge of the hollow spherical shell preferably defines a flat surface. The lateral tilting block in this case is determined by the edge of the hollow spherical shell, with the tilting block in the anterior/posterior direction being determined by the height of the tab-shaped overlap in the corresponding cut. In this development, various maximum angles of tilt are consequently possible in both the lateral and the anterior/posterior directions.

Possibly, an elastic element is additionally provided between the first and second prosthesis plates, with said elastic element effecting a cushioning of the movement of the two prosthesis plates with respect to each other. At the same time, it is possible to form the elastic element so that it essentially encases the coupling elements, so that there is a cushioning of the tilting movements of the two prosthesis plates with respect to each other in all directions.

The elastic cushioning element that encases the coupling element in this case is subjected to pressure, always in areas in which there is a reduction in the distance between the prosthesis plates as a result of the tilting movement of the two prosthesis plates. The elastic element can only be attached to one prosthesis plate, so that there is only a pressure stress on the elastic element, but no tensile stress. The elastic element may be attached to both prosthesis plates, as a result of which there is, on the one hand, a pressure stress and a tensile stress for optimal cushioning effect, while at the same time, a cushioning of the rotational movement also occurs. Finally, with this embodiment, there is also the possibility of sealing the coupling element off from the surroundings. While this may not appear urgently necessary for physiological reasons, because the intervertebral area is largely free of bodily material and bodily fluids, this approach would nevertheless allow the achievement of an even more advanced improvement and reliability of the coupling system.

In a further embodiment, each of the areas in which, when there is a tilting movement both in the lateral and the posterior and anterior directions, elements of the first prosthesis plate or the coupling elements formed thereupon hit against areas of the second prosthesis plate or the corresponding coupling elements formed thereupon when there is maximum tilting, is equipped with block cushioning means, which preferably are manufactured from a stratiform elastic material. In this way, excess mechanical stresses on the material of the prosthesis plates and on that of the coupling elements, which can possibly lead to damage or splintering, are avoided, and any sounds that may arise when the parts hit together, and that could prove to be disturbing to the patient, are ruled out.

The first and second prosthesis plates are, for instance, manufactured of a cobalt-chromium alloy, titanium, or implant steel. By coating the sides of the prosthesis plates facing the bones with titanium plasma and/or a hydroxylapatite coating, improved osseointegration of the prosthesis plates is ensured. To reduce the abrasion of the coupling elements, they can also be manufactured from ceramic.

In a further embodiment, the prosthesis plates are produced in such a way that a convex form or curvature is provided on each of the sides facing away from the coupling elements. In this way, it is possible to insert the intervertebral implant into the intervertebral space in a manner that is particularly easy from a surgical point of view. In comparison to the pins or burrs that usually project very far and that are used for intervertebral implants according to the state of the art and that engage deeply into the respective neighbouring vertebral body, the anchoring of the prosthesis plate, said anchoring being formed only as a convex shape or curvature, to the vertebral body still produces sufficient certainty of the attachment of the prosthesis plate to the skeleton, while still allowing placement that is considerably easier and that also allows a revision operation, in which the intervertebral implant can be removed and replaced with a replacement product. In an embodiment, the convexly shaped surface of the respective prosthesis plate is adapted to the inner contour of the vertebral body in the intervertebral area. From a surgical point of view, the intervertebral implant according to the invention consequently can be inserted very easily, by strongly curving the spinal column of the patient in the corresponding area during the course of the surgery, as a result of which a gap, which opens outwards, forms in the relevant intervertebral area, whereby the intervertebral implant according to the invention, with the convexly shaped exterior surface of the respective prosthesis plate, can easily be inserted by the surgeon into said gap, while prosthesis plates according to the state of the art must be inserted individually, and the coupling elements subsequently have to be put into place separately between the two prosthesis plates, which have already been anchored in the spinal column. The development of the prosthesis plates according to the invention in this embodiment therefore allows a single-piece formation of the coupling elements on the respective prosthesis plates, so that in this way, in turn, simplification of the intervertebral implant is possible, which contributes to an improvement in the reliability of the implant and to a reduction in the costs for manufacturing the implant.

According to a further embodiment, the surface elements of the coupling elements, said surface elements being conjugate to each other, are suitably formed so that, in the event of a tilt of the first and second prosthesis plates with respect to each other, there is a corresponding translational movement at the same time. In particular, this formation of the conjugate surface elements of the coupling elements includes the use of a spherical cap, which is only partially held in a corresponding hollow spherical shell surface. In the case of tilting of the two prosthesis plates with respect to each other, the hollow spherical shell surface then wanders across the spherical cap along the surface of the spherical cap, whereby at the same time, a certain translational movement of the prosthesis plate formed on the hollow spherical shell surface occurs with respect to the opposite prosthesis plate with simultaneous tipping.

The translational movement can be adjusted to the physiologically suitable dimension by means of the selection of the radius of the spherical cap. The radius of the spherical cap may be 0.5 to 0.8 times the anterior/posterior extension of the first prosthesis plate.

Finally, in a further embodiment, a lining is provided between the spherical cap and the seat in the hollow spherical shell surface, whereby this lining in turn can consist of a thin, stratiform elastic material.

Each of the previously mentioned characteristics described as an embodiment can be provided in the intervertebral implant according to the invention individually or they can be combined in any way.

The invention is explained in more detail and described in the following description in conjunction with the appended drawings on the basis of exemplary embodiments. The invention shall not be limited to these embodiments or those defined in the appended claims, but shall encompass any alternatives or modifications lying within the spirit and scope of this disclosure.

### BRIEF DESCRIPTION OF THE DRAWINGS

The drawings, which illustrate the invention by way of example, show the following:

FIG. 1 is a three-dimensional general view of an intervertebral implant according to the invention,

FIG. 2 is an exploded representation of the intervertebral implant shown in FIG. 1,

FIG. 3 is a perspective view of the intervertebral implant according to the invention, in the opened state,

FIG. 4 is a cross-sectional view taken along the line IV-IV from FIG. 1,

FIG. 5 is a cross-sectional view taken along the line V-V from FIG. 1 with anterior tipping,

FIG. 6 is an enlarged depiction of the sub-area X in FIG. 4,

FIG. 7 is a side-view from the anterior direction of the intervertebral implant according to the invention, and

FIG. 8 is a side-view from the posterior direction with tipping and rotation of the intervertebral implant.

### DETAILED DESCRIPTION

The invention is explained in more detail and described in the following using the figures indicated in the preceding. By way of introduction, first the physiological background for the intervertebral implant is explained.

The intervertebral implant according to the invention serves as a replacement for an intervertebral disc. The body's own intervertebral disc represents a structure with a colloidal core and a fibrous ring which are present between two end plates. The intervertebral disc lies between two vertebral bodies and is consequently a part of a motion segment which physiologically acts as the basic element of the motion and stability system of the spinal column. The intervertebral disc, if intact, has the function of stabilizing the spinal column, controlling the mobility of the motion segment, which is brought about by the special construction of the fibre ring, maintaining the distance between the vertebral bodies, whereby this function also produces the control of the mobility, and a cushioning in the event of axial stresses on the spinal column. The natural intervertebral disc consequently imparts a limited and cushioned relative movement between two vertebral bodies with respect to a tipping and also with respect to axial rotation. The mobility of the motion system changes throughout the course of a life, however. While bending, stretching and lateral movement become limited in the course of intervertebral disc degeneration, the rotational movement increases in the course of the intervertebral disc degeneration process.

During the development of the present invention, it was recognized that particularly the rotational movement has a special significance for the anatomically and physiologically correct mobility of the motion system. The physiological control of the movement possibilities of the motion segment in a combination of the intervertebral discs, vertebral joint and ligament complex, as well as musculature, is therefore also necessary in view of an artificial implant of an intervertebral prosthesis.

The intervertebral prostheses known from the state of the art do not produce any sufficient control over the motion segment in the spinal column. One problem, in particular, is the fact that the existing intervertebral implants allow too much mobility, and consequently excess stress on the ligaments, which may already have been reduced in number due to the operation and may already have been changed due to wear.

The approach according to the invention now consists of providing an intervertebral prosthesis as an intervertebral disc replacement in the skeleton of the human body, whereby said prosthesis produces controlled mobility for protecting the ligament system of both the affected intervertebral area and the ligament, skeleton and muscle systems involved in the physiological motion sequences. Therefore the control of the mobility is of great importance in the design of an intervertebral disc replacement, particularly because an intervertebral prosthesis is used as an intervertebral disc replacement in the event of degenerative changes. Consequently, a significantly improved implant is created by the intervertebral prosthesis according to the invention, because significantly limited rotational mobility is created in the motion segment provided with the intervertebral implant according to the invention. This leads to relief of the ligaments involved and support for the vertebral joints, which were clearly overtaxed in the case of models which are known from the state of the art and which have full rotational mobility. This finally leads to better long-term stability of the entire motion segment. The intervertebral prosthesis according to the invention, as an intervertebral disc replacement, has the ability of taking part in the interplay with the vertebral joints, in order to ensure a harmonious motion sequence. The intervertebral prosthesis according to the invention creates both the physiological motion sequence and also a cushioning function of axial movements in the spinal column when a cushioning inlay is additionally used in the coupling system. Because of the formation of the coupling element as a spherical cap which engages in a hollow spherical shell surface without full encompassment of the spherical cap, a translational movement is also created, along with a tipping/tilting movement.

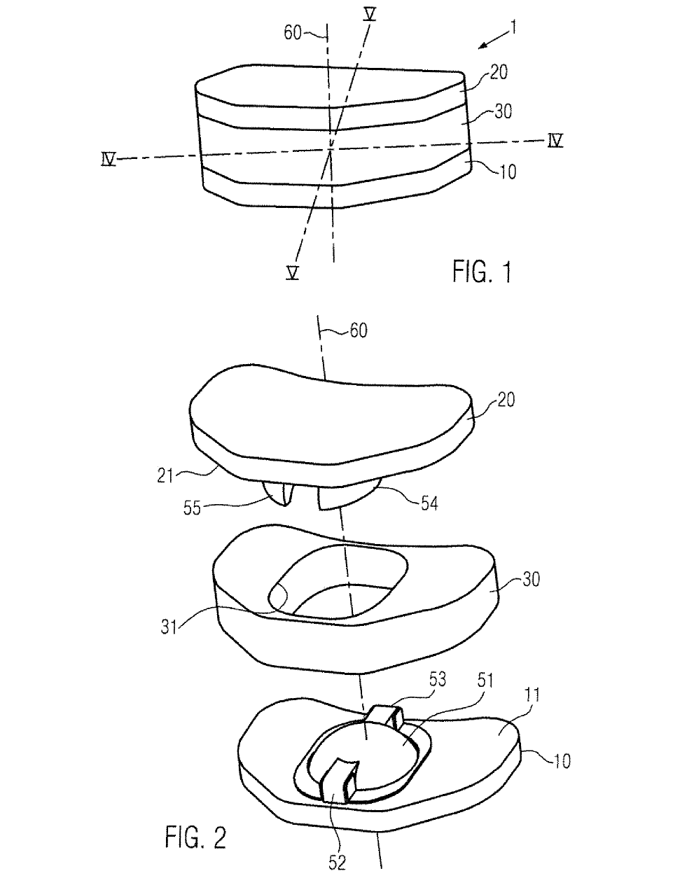

FIG. 1 shows the intervertebral prosthesis 1 according to the invention in the assembled state. The intervertebral prosthesis 1 comprises a first prosthesis plate 10 and a second prosthesis plate 20. An elastic cushioning element 30 is provided between the first and second prosthesis plates. In the untipped state, the first prosthesis shell 10, the second prosthesis shell 20 and the elastic cushioning element 30 are fit together in the coupled state without any gap. The elastic cushioning element 30 forms, with the side surfaces of the first and second prosthesis plates 10 and 20, end edges with an essentially flat transition in each case.

The exploded representation of FIG. 2 shows a first part of a coupling element formed on to the prosthesis plate 10. This part of the coupling element comprises a spherical cap 51, on which the two tab-like overlaps 52 and 53 are formed at the sides. A further part of the coupling element is formed on to the second prosthesis plate 20, with said part having a hollow spherical shell 54. The spherical cap 51 and the hollow spherical shell 54 form surface elements that are conjugate to one another. A pair of cuts 55 and 56 are formed in the shell 54 for holding the tab-shaped overlaps 52 and 53. The size of the cuts 55, 56 is adapted to the tab-shaped overlaps 52 and 53 in such a way that in the coupled state, the tab-shaped overlaps 52 and 53 have play that allows rotational twisting of one prosthesis plate with respect to the other around a predetermined angle, until the side surface of one tab-like overlap hits against the interior side surface of the corresponding cut. In comparison to the size of the tab-like overlaps, the cuts are formed in such a way that, in the coupled state, the first and second prosthesis plates are capable of a maximum rotational movement of 3 degrees around an axis 60 running perpendicularly to the surface of the prosthesis plate 10 or 20. The outer free surface of the spherical cap 51 is dimensioned in such a way that it is larger than the corresponding cavity that is created by the hollow spherical shell 54.

The elastic cushioning element 30 has a thickness that is essentially the same across its length and defines, in a middle area, an opening 31 which allows the coupling elements formed on the first and second prosthesis plates to be held. The thickness of the elastic cushioning element 30 is selected in such a way that when the first and second prosthesis plates are parallel to each other, the cushioning element lies against the interior surfaces 11 and 21 of the first and second base plates 10 and 20 without a gap in between.

The elastic cushioning element can be attached in the first and/or second prosthesis plate by means of special shaping, e.g., attachable nubs (not shown). An attachment of the elastic cushioning element to the first or second prosthesis plate (or both) can also be produced by means of suitable projections on the inner surfaces 11 and 21 of the first and second prosthesis plates, e.g., by means of dovetail-like formations, mushroom-shaped formations or the like, which can be fixed in place in correspondingly suitable cuts in the elastic element (not shown in the figures).

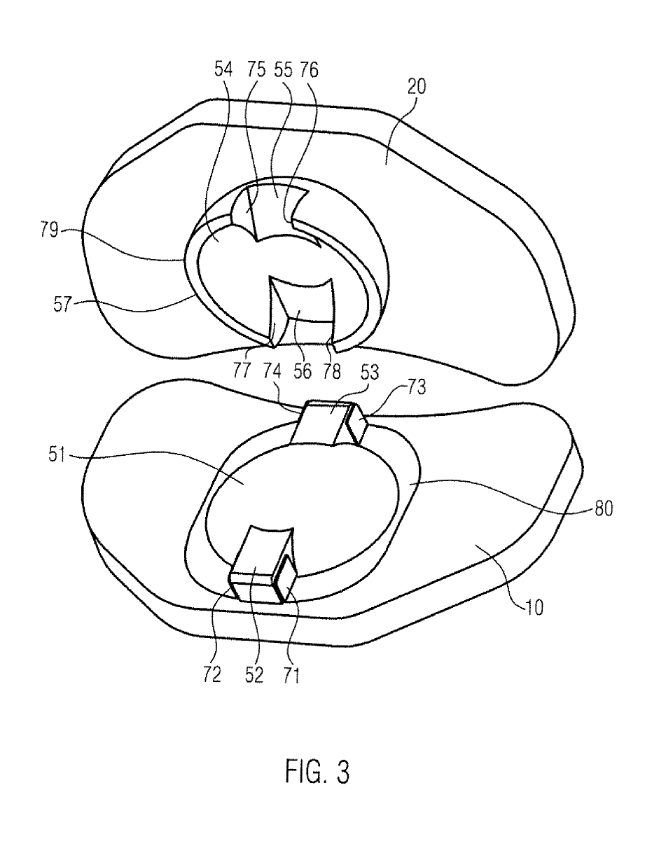

FIG. 3 shows additional cushioning elements in the area of the blocking surfaces of the movement restriction means. Flat elastic cushioning overlays 71 and 72, as well as 73 and 74, are provided on the first prosthesis plate 10 on the side surfaces of each of the tab-shaped overlaps 52 and 53, whereby these overlays serve as rotational blocks to restrict the rotational movement. In the same way, a flat elastic cushioning coating 75, 76, 77, 78 can be provided in each of the side inner surfaces of the cuts 55 and 56 of the hollow spherical shell 54 formed on the second prosthesis plate 20. A flat elastic cushioning overlay 79 is furthermore provided on the frontal edge 57 of the hollow spherical shell 54. A flat elastic cushioning overlay 80 can additionally subsequently be present on the first prosthesis plate 10 at the side of the spherical cap 51. The elastic cushioning overlays 79 and 80 cushion the blocking surfaces elastically in the case of a tilting block.

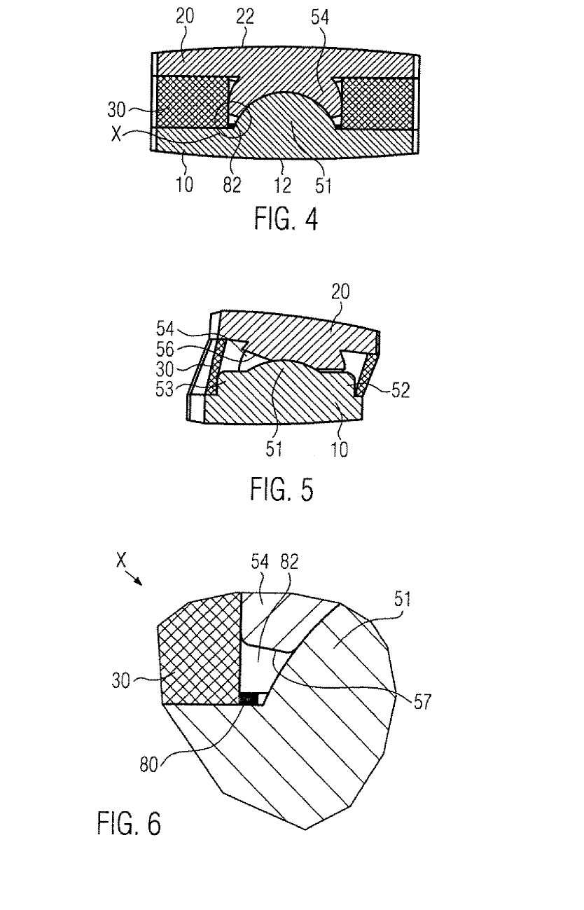

The cross-sectional view of FIG. 4 illustrates that the hollow spherical shell 54 holds the spherical cap 51 only partially, so that a gap remains between the frontal edge surface 57 of the hollow spherical shell and the inner surface 11 of the first prosthesis plate. Because of this gap, a tilting of the first prosthesis plate 10 towards the second prosthesis plate 20 is possible by a predetermined value, which is restricted to a maximum value of 10 degrees in the lateral direction in a preferred embodiment.

FIG. 5 shows the intervertebral prosthesis according to the invention in a side cut, which illustrates the translational displacement and tilting of the first and second prosthesis plates with respect to each other in the event of anterior/posterior bending. As was already to be seen in FIG. 2, the considerably thinner development of the walls formed by the opening 31 in the elastic cushioning element 30 in the anterior and posterior position can be seen in comparison to the lateral wall thickness in the sectional view of FIG. 5. Furthermore to be seen is the movement block with maximum tilting of the first and second prosthesis plates 10 and 20 with respect to each other in the event of forward bending. Because of the hollow spherical shell which glides towards the anterior (towards the right in FIG. 5) on the spherical cap, there is overall also a translation in the anterior direction of the second prosthesis plate with respect to the first prosthesis plate. The internal front faces of the cut 55 and 56 in the hollow spherical shell 54 are sloped in such a way that there is a flat arrangement on the upper top surfaces of the tab-shaped overlaps 52 and 53. The maximum tipping of the first and second prosthesis plates with respect to each other in the anterior and posterior direction, meaning forwards bending and backwards stretching, is determined by the support of the internal front faces of the cuts 55 and 56 on the upper top surfaces of the tab-shaped overlaps 52 and 53. In the example shown, the maximum bending is 10 degrees. The maximum bending can be between 10 and 15 degrees, depending on in which intervertebral area in the spinal column the intervertebral prosthesis is to be inserted. The maximum lateral bending, which is determined by the support of the edge 57 of the hollow spherical shell and the inner surface of the first prosthesis plate, can preferably be between 5 and 6 degrees, likewise depending on the insertion point in the spinal column.

As can be seen in FIG. 4, the outer surfaces 12 and 22 of the first and second prosthesis plates 10 and 20 are formed with a convex shape, said outer surfaces 12 and 22 lying opposite the coupling elements. Due to this convexity, a portion of the volume of the first and second prosthesis plates 10 and 20 lies within the bone edge of the adjoining vertebrae if the intervertebral prosthesis is inserted into the skeleton as an intervertebral disc replacement. In this way, a self-locking effect of the intervertebral prosthesis according to the invention is achieved. The convex form or curvature of the outer surfaces 12 and 22 of the first and second prosthesis plates is advantageously adapted to the anatomical shaping of the inner vertebra surfaces by means of a complementary inner surface topology (not shown). In this way, an even better fit of the prosthesis plates in the intervertebral area is guaranteed.

In FIG. 6, section X (shown in FIG. 4) is depicted in an enlarged view and shows the gap 82 between the edge 57 of the hollow spherical shell and the inner surface 11 of the first prosthesis plate. The frontal edge 57 of the hollow spherical shell 54 is sloped in the direction towards the inner surface of the second prosthesis plate so that in the event of full bending and blocking of the frontal edge surface 57 on the cushioning element 80 on the inner surface of the first prosthesis plate 10, a support across the entire surface, with lateral displacement of the elastic cushioning element 30, takes place.

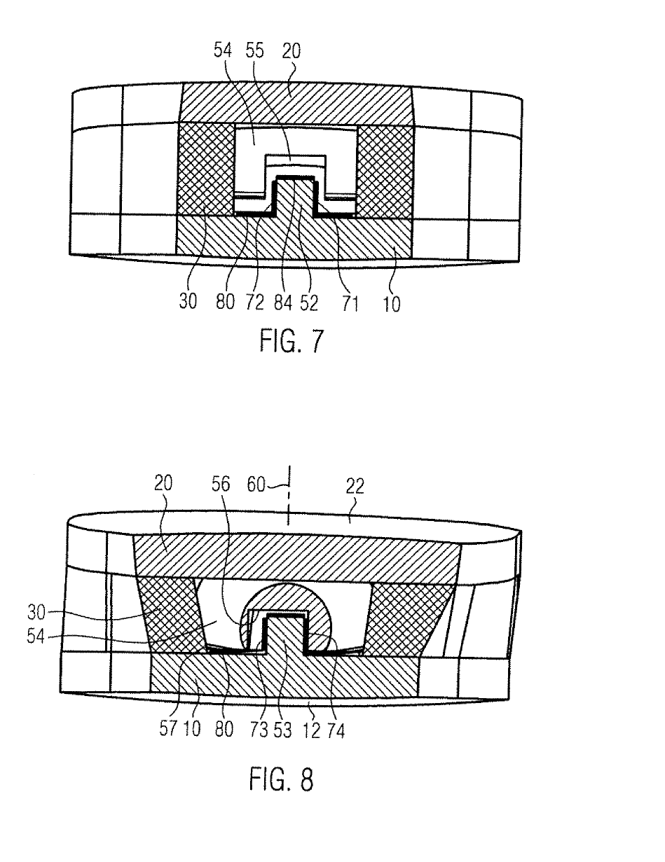

FIG. 7 shows a side-view of the intervertebral prosthesis from the front. In this depiction, a further flat blocking cushion 84 is formed on the upper blocking surface of the tab-shaped overlap 52. In the embodiment shown, in the event of maximum tilting towards the anterior by the two prosthesis plates with respect to one another, there is a blocking both on the upper blocking surface 84 of the tab-shaped overlap 52 and on the inner surface of the prosthesis plate 10 laterally adjacent to the tab-shaped overlap, on which the cushioning elements 80 are likewise present. The blocking can also be achieved, however, solely through the restriction of the block on the upper tab surface on which the cushioning element 84 is provided. In this case there would be no limitation of the maximum stop angle that could be adjusted in various directions, so that a different maximum angle of tilt could be adjusted for each of the angles between anterior/posterior and lateral left and right. When the upper surface of the tab-shaped overlap is used as the sole blocking surface for the end restriction of the maximum angle of tilt in the anterior and posterior directions, it is advantageous to form the upper surface of the tab-shaped overlap as well as the inner surface of the corresponding cut accordingly curved, in order furthermore to create extensive support surfaces in the blocking area when a lateral tipping occurs at the same time.

FIG. 8 shows a side-view of the intervertebral prosthesis according to the invention, from the posterior. In the embodiment shown, the first prosthesis plate 10 is moreover rotated with respect to the second prosthesis plate 20 by the maximum rotational angle around the axis 60 running perpendicular to the prosthesis plates. At the same time, the second prosthesis plate 20 is tipped towards the posterior direction with respect to the first prosthesis plate 10, so that the frontal edge 57 of the hollow spherical shell 54 lies on the flat blocking cushion 80 on both sides of the tab-shaped overlap 53. The flat blocking cushion 74 at the side of the tab-shaped overlap 53 lies on the inner surface of the cut 56 in the hollow spherical shell 54. The cushioning element 30 is subjected to pressure on both sides of the coupling elements in this configuration.

While the preceding described a preferred embodiment of the invention, it is apparent to the person skilled in the art that the invention is not fundamentally restricted to the shown mechanical solution for movement restriction or to coupling of the two prosthesis plates. For example, the tabs can be arranged in the hollow spherical shell, while corresponding cuts are provided in the spherical cap. Instead of pairs of cuts on the edge and tab-shaped overlaps on the respective coupling elements, it would also be possible to have individual cuts and tab-shaped projections centrally arranged on the spherical cap and in the hollow spherical shell surface for this purpose. It is apparent to the person skilled in the art that the indicated shape of the tabs and cuts as essentially in the shape of rectangular blocks is not essential for the invention, but instead other types of the shaping of the tabs and cuts are conceivable, as long as an asymmetry is present for restricting the rotational movement of the two prosthesis plates with respect to each other.

The movement restriction elements can also be formed or developed in such a way that they are separate from the coupling elements on the first and second prosthesis plates.

1. An intervertebral prosthesis for utilisation as an intervertebral disc replacement in the skeleton of the human body, said prosthesis comprising:
a first prosthesis plate and a second prosthesis plate, wherein on both the first and second prosthesis plates, coupling elements with surface elements conjugate to each other are formed, wherein said coupling elements, in the coupled state, allow a rotation as well as a tilting movement in the lateral as well as in the posterior and anterior directions with respect to an axis running essentially perpendicularly to an extension of said first and second plates, and
movement restriction means for restricting the rotational movement to a predetermined maximum value.

2. An intervertebral prosthesis according to claim 1 wherein said restriction means restricts the rotational movement to a rotational angle of 5°.

3. An intervertebral prosthesis according to claim 1 wherein said restriction means restricts the rotational movement to a rotational angle of 3°.

4. An intervertebral prosthesis according to claim 1 wherein said conjugate surface elements of said coupling elements comprise a spherical cap which is provided on said first prosthesis plate and a matching hollow spherical shell surface which is affixed to said second prosthesis plate.

5. An intervertebral prosthesis according to claim 4, wherein at least one tab-shaped overlap is formed on the base area of said spherical cap, said overlap engaging, in the coupled state, in a cut that restricts the corresponding predetermined maximum value of the rotational angle and that is formed in said hollow spherical shell surface.

6. An intervertebral prosthesis according to claim 5, wherein two overlaps, which lie opposite each other, are provided on said spherical cap, with said overlaps engaging in two corresponding cuts, which lie opposite each other, in said spherical shell in the coupled state.

7. An intervertebral prosthesis according to claim 1 wherein said movement restriction means restrict the tilting movement in the posterior and anterior direction to a predetermined value of 15°.

8. An intervertebral prosthesis according to claim 1 wherein said coupling elements are suitably formed to restrict the tilting movement in the lateral direction to a predetermined maximum value in the coupled state.

9. An intervertebral prosthesis according to claim 8 wherein said coupling elements restrict the maximum value of the lateral tilting movement to 6°.

10. An intervertebral prosthesis according to claim 1 wherein said conjugate surface elements of said coupling elements comprise a spherical cap which is provided on said first prosthesis plate and a matching hollow spherical shell surface which is affixed to said second prosthesis plate, wherein said spherical cap is only partially held in said hollow spherical shell surface.

11. An intervertebral prosthesis according to claim 10 wherein front faces of said hollow spherical shell are formed in such a way that each is sloped in the direction towards said prosthesis plate.

12. An intervertebral prosthesis according to claim 1 further comprising an elastic element provided between said first and second prosthesis plates.

13. An intervertebral prosthesis according to claim 12 wherein said elastic element encases said coupling elements.

14. An intervertebral prosthesis according to claim 12 wherein said elastic element is attached to at least one of said first and second prosthesis plates.

15. An intervertebral prosthesis according to claim 1 further comprising blocking cushioning means provided in the area of the blocking of at least one of the rotational and the tilting movement.

16. An intervertebral prosthesis according to claim 15 wherein said blocking cushioning means comprise a stratiform elastic material.

17. An intervertebral prosthesis according to claim 1 wherein said first and second prosthesis plates comprise cobalt-chromium alloy.

18. An intervertebral prosthesis according to claim 1 wherein said first and second prosthesis plates comprise titanium.

19. An intervertebral prosthesis according to claim 1 wherein said first and second prosthesis plates comprise ceramic.

20. An intervertebral prosthesis according to claim 1 wherein said conjugate surface elements are coated at least partially with at least one of titanium plasma coating and hydroxylapatite coating.

21. An intervertebral prosthesis according to claim 1 wherein at least one of said first and second prosthesis plates has a convex shape on the side facing away from said coupling elements.

22. An intervertebral prosthesis according to claim 21 wherein said first and second prosthesis plates, on the sides facing away from the coupling elements, have a surface topology designed according to the intervertebral surfaces of the vertebrae and complementary to them.

23. An intervertebral prosthesis according to claim 1 wherein said conjugate surface elements of said coupling elements are suitably formed to allow a translational movement of the first and second prosthesis plates in the posterior/anterior direction in the coupled state.

24. An intervertebral prosthesis according to claim 1 wherein each of the two parts of said coupling elements is formed integrally with said first and second prosthesis plates, respectively.
